(19) United States
(12) Patent Application Publication
(10) Pub. No.: US 2019/0159712 A1
(43) Pub. Date: May 30, 2019

(54) BRAIN FUNCTION DECODING PROCESS AND SYSTEM

(71) Applicant: Donald H. Marks, Wharton, NJ (US)

(72) Inventor: Donald H. Marks, Wharton, NJ (US)

(21) Appl. No.: 16/265,045

(22) Filed: Feb. 1, 2019

Related U.S. Application Data
(63) Continuation-in-part of application No. 12/731,264, filed on Mar. 25, 2010.
(60) Provisional application No. 61/164,724, filed on Mar. 30, 2009.

(51) Int. Cl. A61B 5/16; A61B 5/00; A61B 5/055; G01R 33/48

(52) U.S. Cl. CPC A61B 5/16; A61B 5/0042; A61B 5/4064; G01R 33/4806; A61B 5/055

(57) ABSTRACT

A process of correlating cognitive response to a stimulus is provided by collecting biometric data with a camera having a microprocessor for modifying an image by localizing eyes within a face in the image, collecting neural activity data from a subject while exposed to a stimulus and mapping neural activation in response to the stimulus onto neuronal structure of a subject brain. A unique three-dimensional cognitive engram representative of the neural activity caused by the stimulus is then constructed. The cognitive engram is then compiled in a database containing a plurality of engrams, each of which is associated with a different stimulus for interpreting cognitive response. Then, each unique three-dimensional cognitive engram stored in the database is correlated with the biometric data representative of a stimulus.

### CROSS REFERENCE TO RELATED APPLICATIONS

This application is a continuation in part of U.S. application Ser. No. 12/731,264 filed Mar. 25, 2010 that in turn is a continuation in part of U.S. application Ser. No. 12/626,208 filed Nov. 25, 2009, now U.S. Pat. No. 7,627,370 issued Dec. 1, 2009, which claims priority to U.S. Provisional Application No. 60/620,507 filed Oct. 20, 2004, now abandoned. This application further claims priority to U.S. Provisional Application No. 61/164,724 filed Mar. 30, 2009, now abandoned, the entire contents of these priority applications are incorporated herein by reference.

### FIELD OF THE INVENTION

The present invention in general relates to analysis of neuroimaging data and in particular to correlating facial recognition data from neuroimaging with facial recognition data obtained from a surveillance camera.

### BACKGROUND OF THE INVENTION

Scientific research has demonstrated that memory and consciousness reside in the organization of neurons, their interconnections, charges on the cell surfaces, intracellular and extracellular proteins and other molecules, and other factors. The ability to understand the molecular and cellular mechanism of memory and consciousness, the sequences, the charges, and how they relate to memory and consciousness is in very early development; the capability to record and analyze these data in detail currently exists.

Great strides have been made in the area of functional and structural imaging of the human brain. The ability to interpret and correlate brain imaging information and the stimuli that result in memories, thoughts and concepts is in various stages of research and development. The technology for imaging the macroscopic, microscopic and molecular structure of individual human brains, through techniques such as Computed Axial Tomography (CT or CAT), functional Magnetic Resonance Imaging (fMRI), Positron Emission Tomography (PET), electroencephalography (EEG), and Magnetoencephalography (MEG), singly and in combination (collectively referred hereafter as neuroimaging technologies), is continually improving. Recent progress in improved imaging resolution and advancing computational analysis of the data has led to evolving understanding of where thoughts are encoded in the brain.

Kim et al. (2003) used Blood Oxygenation Level-Dependent (BOLD)-based functional MRI (fMRI) and diffusion tensor imaging (DTI) on the cat visual cortex to allow three-dimensional fiber reconstruction, to construct a map of the axonal circuitry underlying visual information processing. Rueckert et al. (2003) used the concept of statistical deformation modeling to construct average models of neuroanatomy and variability of 25 different human subjects. With newer MRI machines with stronger (3 Tesla and above) magnetic fields and improved software, cellular resolution is now being attempted.

Zeineh et al. (2003) used high-resolution fMRI to study the process of encoding and retrieval of memories of names associated with faces, in the medial temporal lobe (MTL) of the human brain and its subregions. The cornu ammonis and the dentate gyrus regions of the brain were active relative to baseline only during encoding, and this activity decreased as associations were learned. Activity in the subiculum region showed the same temporal decline, but primarily during retrieval. Zeineh and colleagues evaluated changes in the blood oxygen level-dependent (BOLD) response, reflecting neural activity, within different substructures of the MTL as subjects progressively learned new associations. These researchers developed techniques to acquire high-resolution structural (0.4 by 0.4 mm) and functional (1.6 by 1.6 mm) MRI data and to localize functional activity precisely within the substructures of the hippocampus.

Zeineh et al. manipulated neuroimaging imaging data to mathematically represent an “unfolding” of the hippocampal cortex, which revealed the entirety of each hippocampal subregion (within the resolution restrictions of the equipment) and adjacent neocortical regions (parahippocampal, entorhinal, perirhinal, and fusiform) in a single plane, or “flat map” representation. Boundaries were demarcated between the architectonic subregions on the high-resolution structural MR images. The white matter and CSF throughout the MTL were segmented and separated out, retaining only the gray matter sheath. Gray matter was then computationally extracted and flattened (similar to flattening the globe into a flat map of the world) to project the demarcated boundaries, producing unfolded flattened maps of the hippocampus.

Using these techniques, Zeineh et al. studied ten human subjects, who were scanned while they performed a face-name association task in which a series of unfamiliar (but could have also been familiar) faces were paired with names. Computational warping techniques transformed an individual subject's hippocampal maps to the flat hippocampal template space. The same transformation parameters were then applied to the coregistered functional MRI scans, which delivered high-resolution fMRI data in a standardized flat space. This procedure enabled measuring activity over time in each subregion and to perform powerful group statistics across subjects. Similarly, Cognitive Engineering, LLC is creating data models of concepts, emotions, and of specific visual objects, such as a face.

Zeineh and colleagues were able to show a strong, parametric correlation between activity in specific brain areas and the storage of new associations. As the number of new associations learned decreased from block to block, activity in these regions fell in parallel. They also found a similarly strong relationship between activity in the subiculum and retrieval of newly learned associations.

Because subjects vary in the anatomy of their MTLs, Zeineh et al. constructed a template representing the typical anatomy of the subject population by averaging together the individual demarcation boundaries across subjects. This is somewhat analogous to the work of Rueckert (automatic construction of three-dimensional statistical deformation models (SDM) of the brain using nonrigid registration). Using a group-averaged incremental performance curve, the researchers regressed MR signal intensity in each pixel and each subject with two waveforms reflecting either performance during learning or performance during retrieval, and then statistically tested whether the slope of each regression for a given pixel was on average different from zero.

In summary, Zeineh et al. identified mnemonic properties of different subregions within the hippocampal circuitry as human subjects learned to associate names with faces. The cornu ammonis (CA) fields 2 and 3 and the dentate gyrus were active relative to baseline only during encoding, and this activity decreased as associations were learned. Activity in the subiculum showed the same temporal decline, but primarily during retrieval.

Ishai and Ungerleider (1999) identified, using fMRI, three bilateral regions in the ventral temporal cortex that responded preferentially to faces, houses, and chairs. In a follow-up report (Ishai 2000) they demonstrated differential patterns of activation, similar to those seen in the ventral temporal cortex, in the bilateral regions of the ventral occipital cortex. They also found category-related responses in the dorsal occipital cortex and in the superior temporal sulcus. Moreover, rather than activating discrete, segregated areas, each category was associated with its own differential pattern of response across a broad expanse of cortex.

The distributed patterns of response were similar across tasks (passive viewing, delayed matching) and presentation formats (photographs, line drawings). Ishai et al. (2000) proposed that the representation of objects in the ventral visual pathway, including both occipital and temporal regions, is not restricted to small, highly selective patches of cortex but, instead, is a distributed representation of information about object form. Within this distributed system, the representation of faces appears to be less extensive as compared to the representations of non-face objects.

Koechlin et al. (2003) showed that the lateral pre-frontal cortex (PFC) of the brain is organized as a cascade of executive (controlling) processes from premotor to anterior PFC regions. These processes control behavior according to stimuli, the present perceptual context, and the temporal episode in which stimuli occur, respectively. Koechlin et al.'s results support a unified modular model of cognitive control that describes the overall functional organization of the human lateral PFC and has basic methodological and theoretical implications.

Fan et al. (2003) studied whether source information, item information, or both are required at the time of memory retrieval. Two sources were used in a factorial design in which the main effect of source and item retrieval, along with their interaction, could be measured by fMRI activations. They found that when source information was required at retrieval, the left frontal lobe showed significant activation but not when item retrieval was required. Activation of the hippocampal section of the brain showed no difference between source and item retrieval. Fan et al.'s data supports a larger role for the frontal lobes in encoding and retrieval of source information.

Nielson et al. (2004), utilizing statistical data mining of a neuroimaging database, located associations between various words/text and brain locations. This provided an understanding of how the brain associates words indicative of cognitive function.

It appears that in all of the studies to date, neuroimaging researchers have mapped gross brain functional activation with various macroscopic regions of the brain. There has not been an attempt to understand how individual brain imaging is directly linked to the concepts that form the basis of thought.

Givens et al. (1999) reviewed their and other data using EEG to study higher brain function. They emphasized the ability of more modern EEG studies to complement functional neuroimaging techniques. The invention disclosed herein may utilize multiple simultaneous neuroimaging techniques, including supplementation by EEG, during the construction of some data sets.

Suppes et al. (1998) studied the ability of recordings of electrical and magnetic brain waves of two subjects to recognize which one of twelve sentences or seven words auditorily presented was processed. The analysis consisted of averaging over trials to create prototypes and test samples, to each of which a Fourier transform was applied, followed by filtering and an inverse transformation to the time domain. The filters used were optimal predictive filters, selected for each subject. A still further improvement was obtained by taking differences between recordings of two electrodes to obtain bipolar pairs that then were used for the same analysis. Recognition rates, based on a least-squares criterion, varied, but the best were above 90%. The first words of prototypes of sentences also were cut and pasted to test, at least partially, the invariance of a word's brain wave in different sentence contexts. The best result was above 80% correct recognition. Test samples made up only of individual trials also were analyzed. The best result was 134 correct of 288 (47%), compared to the expected recognition number by chance (24, or 8.3%).

Hartley and Speer (2000) reviewed functional neuroimaging data of memory, and discussed progress in understanding memory systems. Rugg et al. (2002) reviewed fMRI use to study episodic memory in humans. The data they review, while impressive, does not localize below general brain region areas of activation.

Fujii et al. (2002) used PET to image normal volunteers engaged in deep (semantic) or shallow (phonological) processing of new or repeated words. Their results showed that deep processing, compared with shallow processing, resulted in significantly better recognition performance and that this effect was associated with activation of various brain areas. Regions directly relevant to episodic memory encoding were located in the anterior part of the parahippocampal gyrus, inferior frontal gyrus, supramarginal gyrus, anterior cingulate gyrus, and medial frontal lobe in the left hemisphere. The authors concluded that several regions, including the medial temporal lobe, play a role in episodic memory encoding.

Zhang et al. (2003) studied the involvement of frontal cortex in accessing and evaluating information in working memory using a variant of a Sternberg paradigm and comparing brain activations between positive and negative responses (known to differentially tax access/evaluation processes). Test subjects remembered two trigrams in each trial and were then cued to discard one of them and maintain the other one as the target set. After a delay, a probe letter was presented and participants made decisions about whether or not it was in the target set. Several frontal areas—anterior cingulate (BA32), middle frontal gyrus (bilateral BA9, right BA10, and right BA46), and left inferior frontal gyrus (BA44/45)—showed increased activity when participants made correct negative responses relative to when they made correct positive responses. No areas activated significantly more for the positive responses than for the negative responses. The authors suggested that the multiple frontal areas involved in the test phase of this task may reflect several component processes that underlie more general frontal functions.

Schmithorst and Holland (2004) used fMRI to study neural correlates of the link between formal musical training and mathematics performance in normal adults. Musical training was associated with increased activation in the left fusiform gyms and prefrontal cortex areas of the brain, and decreased activation in visual association areas and the left inferior parietal lobule of the brain during a mathematical task. The authors hypothesized that the correlation between musical training and math proficiency may be associated with improved working memory performance and an increased abstract representation of numerical quantities.

Lanius et al. (2004) used both 4-T fMRI and functional connectivity analyses to assess interregional brain activity correlations during the recall of traumatic memories in traumatized subjects with and without posttraumatic stress disorder (PTSD). Comparison of connectivity maps at the right anterior cingulate gyrus brain region for the two groups showed that the subjects without PTSD had greater correlation than the PTSD subjects in the following brain areas: left superior frontal gyrus (Brodmann's area 9), left anterior cingulate gyms (Brodmann's area 32), left striatum (caudate), left parietal lobe (Brodmann's areas 40 and 43), and left insula (Brodmann's area 13). In contrast, the PTSD subjects showed greater correlation than the subjects without PTSD in the right posterior cingulate gyrus (Brodmann's area 29), right caudate, right parietal lobe (Brodmann's areas 7 and 40), and right occipital lobe (Brodmann's area 19). The authors concluded that the differences in brain connectivity between PTSD and comparison subjects may account for the nonverbal nature of traumatic memory recall in PTSD subjects, compared to a more verbal pattern of traumatic memory recall in comparison subjects.

Na et al. (2000) used fMRI to image working memory in humans. Like all studies to date, they were able to determine gross brain areas of activation, but were limited on their resolution. Na et al. assessed activated brain areas during stimulation tasks (item recognition), followed by an activation period. The prefrontal cortex and secondary visual cortex were activated bilaterally by both verbal and visual working memory tasks, and the patterns of activated signals were similar in both tasks. Bilateral prefrontal and superior parietal cortices activated by the visual working memory task may be related to the visual maintenance of objects, representing visual working memory.

Their activation map images of the upper level of the brain showed neither activated signals in the supramarginal gyrus nor lateralization of activated signals in the frontal and parietal lobes. Map image of the middle level of the brain showed no activated signals in the left inferior frontal or temporal gyrus. An activated signal in the prefrontal cortex corresponded to the signal activated during the verbal working memory task. Map image of the lower level of the brain showed bilateral activated signals similar to those seen during the verbal working memory task in the right and left occipital cortices and posterior fusiform gyri.

Zhang et al. (2003) compared brain activations between positive and negative responses (known to differentially tax access/evaluation processes) to investigate the involvement of frontal cortex in accessing and evaluating information in working memory. Participants remembered two trigrams in each trial and were then cued to discard one of them and maintain the other one as the target set. After a delay, a probe letter was presented and participants made decisions about whether or not it was in the target set. Several frontal areas—anterior cingulate (BA32), middle frontal gyrus (bilateral BA9, right BA10, and right BA46), and left inferior frontal gyrus (BA44/45)—showed increased activity when participants made correct negative responses relative to when they made correct positive responses.

Blaizot et al. (2000) used PET data to map the visual recognition memory network in the baboon. Using computerized matching to sample visuomotor control tasks, they matched PET data to that obtained by anatomic MRI images. They found that foci of significant activation were distributed along the following brain areas: ventral occipitotemporal pathway, inferomedial temporal lobe, and orbitofrontal cortex, consistent with activation studies in healthy humans.

In addition to the work of Ishai and of Zenich, referred to above, there are a number of other studies supporting the accessibility of neural data for interpretation of the actual thought processes occurring. Anderson et al. were able to record signals from neurons in monkeys and showed how they were coding for movement, an important step towards creating better prosthetic devices for paralyzed people. The decoded signals enabled the researchers to predict the monkeys' arm movements in tasks in which they thought about reaching for an item without actually doing so. Further, their research suggests that other types of cognitive signals can be decoded from patients.

Past studies on monkeys have shown that information from neurons coding movement instructions can be used to control prosthetic devices. For example, Rhesus monkeys could be taught to control and assimilate a robot arm using signals from their brain. To achieve this, researchers implanted an array of microelectrodes into the frontal and parietal lobes—areas of the brain involved in producing multiple output commands to control complex muscle movements. The faint brain signals from the electrodes were detected and analyzed by a computer system to recognize patterns of signals that represent particular movements by an animal's arm. These signals were translated into similar movements of a robotic arm.

Andersen and colleagues implanted in monkeys arrays of electrodes into areas of the brain that encode the goals of reaching movements rather than controlling movement itself. While the monkeys waited for a cue that told them to reach for an icon flashing on a screen, a computer program interpreted the brain signals recorded by the electrodes. Once the “neuronal code” was cracked, the researchers used the program to decipher the direction that the monkeys were planning to reach for during trials in which they thought about reaching but didn't actually do so. When monkeys remained still while having thoughts that were consistent with requested movements, they received a reward.

At first, the program had trouble matching the monkeys' intentions to the icon's position much more often than chance. As the monkeys practiced thinking about reaching, however, their neural signals became stronger, enabling the program to decode the correct direction more frequently. Eventually, the program could predict the intended direction of the monkeys' reach as much as 67% of the time. When the monkeys knew that accurately thinking about the requested movement would yield a preferred reward, the computer's ability to predict direction improved by as much as 21%. For instance, recording thoughts from speech areas could alleviate the use by those unable to speak (stroke, other neurologic diseases) of more cumbersome letter boards and time-consuming spelling programs, or recordings from emotion centers could provide an online indication of a patient's emotional state.

The overall significance of these studies is that changes in the brain occur during active memory, and these changes can be observed using existing neuroimaging technologies.

Facial recognition by biometric analysis (Hammond 2007) of a picture shows dependence on the structure of the face. Similarly, face representation by fMRI is also dependent on facial geometry. For example, Loffier et al., Nat Neurosci., 2005 October; 8(10):1386-90, incorporated herein by reference, found that neural activation patterns for individual faces are encoded as grouped data. This encoding varied on the direction (facial identity) and distance (distinctiveness) from a standard or prototypical (mean) face. Varying facial geometry (head shape, hairline, internal feature size and placement) caused the corresponding fMRI signal to increase with increasing distance from the mean face. Also, the same neural population responds to faces falling along single identity axes within this space.

Different facial recognition active areas of the brain demonstrate different activation patterns dependent on physical or identity changes. For example, Rotshtein et al., Nat Neurosci., 2005 January; 8(1):107-13, incorporated herein by reference, showed that the inferior occipital gyrus (TOG) demonstrates sensitivity to physical rather than to identity changes in a stimulus image. In contrast, the right fusiform gyrus (FFG) demonstrates sensitivity to identity rather than to physical changes. Bilateral anterior temporal regions show sensitivity to identity change that varies with the subjects' pre-experimental familiarity with the faces.

The brain also brain processes featural information and second-order spatial relations in face identity processing. Rotshtein et al., J Cogn Neurosci., 2007a September; 19(9):1435-52, incorporated herein by reference, used fMRI to measure second-order special relations between facial features including eyes, mouth, and nose. These authors found that feature-dependent effects occurred in the lateral occipital and right fusiform regions of the brain. Spatial relation effects, in contrast, occurred in the bilateral inferior occipital gyrus and right fusiform regions. Overall, these authors found that featural and second-order spatial relation aspects of faces make distinct contributions to behavioral discrimination and recognition. Face features contributed most to face discrimination, whereas second-order spatial relational aspects correlated best with recognition skills.

Using “hybrid” faces, Rotshtein P, et al., Cereb Cortex, 2007b; 17(11):2713-24, incorporated herein by reference, identified repetition and attention as affecting partly overlapping occipitotemporal regions; these did not interact. Changes of high Spatial Frequency SF faces increased responses of the right inferior occipital gyrus (TOG) and left inferior temporal gyrus (ITG), with the latter response being also modulated additively by attention. The bilateral middle occipital gyrus (MOG), in contrast, responded to repetition and attention manipulations of low SF. A common effect of high and low SF repetition was observed in the right fusiform gyrus (FFG). Follow-up connectivity analyses suggested direct influence of the MOG (low SF), IOG, and ITG (high SF) on the FFG responses. Overall, different regions within occipitotemporal cortex extract distinct visual cues at different SF ranges in faces, and the outputs from these separate processes project forward to the right FFG where the different visual cues may converge.

Despite these breakthroughs in understanding how the brain processes visual stimuli, no effective processes or systems have been successful in predicting brain responses to visual stimuli such as a recognized or unrecognized face. There is a long unmet need for non-invasive and humane methods of determining human recognition and truthful responses. Thus, there exists a need to translate neurologic changes occurring upon sensory or physical stimulation into a decipherable code allowing one to effectively read neurologic activity of an individual. There also exists a need for methods of predicting brain responses to recognition or non-recognition of faces and correlating these responses to recognition systems of any two- or three-dimensional image.

### SUMMARY OF THE INVENTION

The present invention provides a process of correlating cognitive response to a stimulus is provided by collecting biometric data with a camera having a microprocessor for modifying an image by localizing eyes within a face in the image, collecting neural activity data from a subject while exposed to a stimulus and mapping neural activation in response to the stimulus onto neuronal structure of a subject brain. A unique three-dimensional cognitive engram representative of the neural activity caused by the stimulus is then constructed. The cognitive engram is then compiled in a database containing a plurality of engrams, each of which is associated with a different stimulus for interpreting cognitive response. Then, each unique three-dimensional cognitive engram stored in the database is correlated with the biometric data representative of a stimulus.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram of an inventive process to determine a cognitive engram;

FIG. 2 The look-up operation of the Rosetta Database, allowing two-way correspondence between brain activation patterns and identification or correlation to the initial stimulation (face, object, emotion, truth state);

FIG. 3 is a schematic illustrating how correlated data on brain activation obtained from functional MR imaging while viewing specific visual stimuli correlates with the biometric data obtained using facial recognition software; and

FIG. 4 illustrates data transmitted from a single camera to a remote or local computer for subsequent comparison and identification.

### DETAILED DESCRIPTION

The present invention has utility in producing a cognitive engram representation of neuroimaging data associated with the neurophysiological changes that are responsible for storage of a specific memory element in the brain. Producing cognitive engrams is optionally from translating or correlating predictive or measured information to information from facial recognition systems. The present invention finds utility in settings such as interrogation, national security, criminal investigation, marketing and pharmacology.

While a memory engram is known to one skilled in the art as a term that refers to the physical changes in the brain that accompany learning, as used herein the term “cognitive engram” is defined as a multidimensional representation of neuroimaging data of neurophysiological changes that are responsible for storage of a specific memory element or concept in the brain. A subset of a cognitive engram is appreciated herein to include truth/deception indications.

A “subject” is defined herein to include a human; non-human mammals such as monkeys, chimps, rats, mice, rabbits, dogs, cats, pigs, sheep and cows; and birds such as pigeons and chickens.

According to the present invention, cognitive engrams are described that capture memories of persons, places, events, concepts, intentions or thoughts from the brain of a subject and as such are readily adapted to discerning hidden agendas, past criminal activities, plans for the future, collaborative names, crime scene locations and other information that for instance is relevant to criminal investigations. It is appreciated that interrogation uses of the present invention are optionally facilitated by exposing a subject to stimulus while the subject is sedated, sleeping, or unconscious. Additionally, application of various stimuli to a given subject is optionally applied directly onto the appropriate receptor organ (ear, eyes, etc.) or via direct brain stimulation.

It is appreciated that conventional software routines are operative herein to construct spatially localized images and correlate concepts and items introduced via subject senses. Such software routines illustratively include subtracting test images from corresponding baseline images, cryptography software, motion direction software that involves use of moving overlap, correlation across various imaging techniques, neural network software, statistical parametric mapping software, analysis of functional neuroimages (AFNI software), holographic routines and combinations thereof.

Gradient imaging is optionally used to fluctuate the magnetic gradient of the MRI signal to enhance the resolution and content of the signal data. PET-MRI image registration is used to correlate images and data in both structural and functional terms. Although such data is obtained from separate scans with separate techniques, the resulting data extracted from the different scans on the same individual afford common structural and functional information. CT-MRI image fusion analysis is also operative herein to correlate images and data with both structural and functional components. This is analogous to FET-MRI image registration. In another embodiment, the comparison of dynamic MRI imaging with dynamic double arterial phase helical computer tomography is used to evaluate the structure and function of blood supply and perfusion and its relation to the encoding of memory. Virtual MRI constructions of finer resolution are appreciated to correlate functionally active regions of a subject brain to specific concept encoding. The resulting patterns are correlated through the stimulus a subject is being subjected to during neuroimaging. In other embodiment, three-dimensional image correlation of CT, magnetic resonance and PET studies are compared to further enhance structural and functional correlations within a subject brain and categorize those correlations with a stimulus or thought.

In some embodiments a functional magnetic resonance imaging in real-time (FIRE) uses sliding-window correlation analysis and reference-vector optimization of imaging data to understand encoding of specific stimulus as concepts within the subject brain in a mechanistic fashion.

In some embodiments arterial spin labeling and dynamic susceptibility weighted contrast enhanced magnetic resonance imaging are used to discern, identify and match two-dimensional brain imaging data to the concepts being stored in a subject brain.

In some embodiments MRS—magnetic resonance spectroscopy—is used to discern, identify and match variations in metabolites in neurons during neural activity to the concepts being stored in a subject brain.

In some embodiments MRA—magnetic resonance angiography—is used to discern, identify and match variations in blood flow during neural activity to the concepts being stored in a subject brain.

In some embodiments comparison of conventional, magnetization transfer and diffusion-tensor magnetic resonance imaging findings is used with whole brain tissue histogram analysis to discern, identify and match two-dimensional brain imaging data to concepts being stored in a subject brain. Additionally, activation areas of a subject brain are shown by groups of continuous voxels. A voxel is a volumetric pixel, a volume element used in neuroimaging analysis. A voxel represents a value on a regular grid in three-dimensional space. As with pixels in a bitmap, voxels themselves do not typically have their position (their coordinates) explicitly encoded along with their values. Instead, the position of a voxel is inferred based upon its position relative to other voxels (i.e., its position in the data structure that makes up a single volumetric image). In contrast to pixels and voxels, points and polygons are often explicitly represented by the coordinates of their vertices. A direct consequence of this difference is that polygons are able to efficiently represent simple 3D structures with lots of empty or homogeneously filled space, while voxels are good at representing regularly sampled spaces that are non-homogeneously filled. Individual voxels are compared between individuals and within the same individual for multiple runs of the same stimulus. Additionally, windowing is optionally used to further simplify analyses such that instead of, for example, simply using maximal intensity by looking only at subject brain regions having for instance greater than 90% activity or only activity between 60% and 80%, a cognitive engram is more rapidly produced.

The net result of these varied image processing techniques that are applied in the generation and analyses of raw data is to discern a data storage pattern within the brain and prepare a Rosetta Database for the identification and matching of brain imaging scan data with a given stimulus without the need for understanding the rules of stimulus encoding within a subject brain.

Optionally, prior to determination of a cognitive engram, image corrections known to the art are performed to remove image noise. Such additional techniques illustratively include correcting for subject movement artifact, reprocessing sequential images, digital noise subtraction, blood oxygen level dependency consideration, statistical deformation modeling (SDM), and analysis algorithms well known to one skilled in the art for MRI data optionally that exploits differences in data signals not requiring functional MRI or blood oxygen level dependent BOLD techniques.

An additional aspect of the present invention is the development of movement artifact overlap harvesting in which additional data removed by software to correct for movement artifact is harvested and re-processed to fill in detail from spatial areas between imaging slices and as such increases the total data set. The inventive technique of movement artifact overlap harvesting spatially plots conventional motion correction artifacts to create partial pseudoslices intermediate between adjacent MRI imaging slices.

There are a number of methods to analyze complex fMRI face activation data illustratively including: sparse logistic regression (SLR) (Yamashita, et al., Neuroimage, 2008; 42(4):1414-29, incorporated herein by reference), feature vectors (Lee, et al., Medical Image Analysis, 2009; 13:392-404, incorporated herein by reference) using a support vector machine algorithm; quantitative receptive-field models (Kay, et al., Nature, 2008; 452(7185):352-5, incorporated herein by reference), multivoxel pattern information analysis (Mur, et al., Soc Cogn Affect Neurosci. 2009 Jan. 17, incorporated herein by reference), direction and distance from a prototypical (mean) face (Lofflier, et al., Nat Neurosci, 2005; 8(10):1386-90, incorporated herein by reference), feature-spacing analyses (Maurer, et al., Neuropsychologia, 2007; 45:1438-1451, incorporated herein by reference), and others, including those mentioned in U.S. Pat. No. 7,627,370, incorporated herein by reference. In essence, three-dimensional activation patterns from wide areas of the brain are formed into patterns which are equated back to the stimulus for the activation (face, object, concept, emotion). Ultimately, all activation patterns in three-dimensional space form unique data sets specific for the object or concept under consideration—Cognitive Engrams (Marks 2007).

The SLR methods of Yamashita, et al., Neuroimage, 2008; 42(4):1414-29, automatically select relevant voxels while estimating their weight parameters for classification. SLR can automatically remove irrelevant voxels and thereby attain higher classification performance than other methods in the presence of many irrelevant voxels. These activated voxels form what can best be described as a cognitive engram, which can be used to predict or decode fMRI activity patterns.

Methods and algorithms for facial recognition or translating an image into code are known in the art. Faces are optionally analyzed and correlated by analyses of physical features (Hammoud, et al., Multi-Sensory Multi-Modal Systems. Springer, 2007, incorporated herein by reference). Relative sizes and distances for facial landmarks such as eyes, nose, ears, chin, and skin texture, among others, are optionally measured. Image data are optionally extracted from camera images, from video streams, or other methods known in the art. Illustrative methods for extracting biometric data from an image are described in WO 2006/023046, the contents of which are incorporated herein by reference.

Illustrative methods for generating biometric data sets from an image illustratively include those produced by Ayonix Co., Ltd., Osaka City, Japan, that described in JP2009237669, those produced by Neven Vision of Santa Monica, Calif., methods described in WO 2006/023046, the contents of which are incorporated herein by reference, or other methods known in the art.

It is appreciated that numerous biometric measurements are illustratively used for generating a biometric data set. These illustratively include: physical distance parameters such as pupil distances, eye to nose distances, ear to ear distances, hairline to eye; or other physical distance; ratiometric information illustratively ratios of ear circumferences, iris sizes, distance from each eye to mouth center, or other ratio; skin texture; iris pattern or changes in coloration and the like; presence or absence of hair; density or other parameter of hair presence or dimension; or other measurement known in the art.

It is appreciated that while the description is directed to facial recognition in general, other images or patterns are similarly operable. Illustratively, images include a hand, foot, scar, whole body or any part thereof. In some embodiments an image is of a whole human with biometric information including dimensions or ratios such as arm length, leg length, height, shoulder levelness, limb or torso angles, and other parameters. Images need not be of a human. Illustratively, biometric data are optionally of a dog, cat, or other non-human animal. Images are optionally of a vehicle or other inanimate object. As such, the word biometric is optionally used to describe characterizable qualities of an object. Similar to a human face, objects such as automobiles have characteristics that are measurable or quantifiable. Just as siblings have unique physical characteristics, so do sisterships, etc. Objects, subjects, or other images are preferably sufficiently distinguishable from similar objects, subjects, or other images so as to be unique in at least one parameter or set of parameters. In some embodiments, an image is of a dog or other domesticated or non-domesticated animal.

A remote biometric data generating camera is also provided. Known electronic cameras, such as commercially available for the purpose of computer analysis of image phenomena, are optionally employed in conjunction with a localized microprocessor. A camera is illustratively a still photographic camera or a video camera. Imaging is optionally performed by a video or television camera, an infrared camera, a CCD or CMOS scanning device, or any other suitable scanning system. Alternatively, a light source such as an infrared (or other) laser is optionally positioned to perform the imaging and a photodetector of reflected (or emitted) light from such source is optionally positioned to detect the variable reflected (or emitted) light and to generate the data. Such camera systems, absent a microprocessor are described in U.S. Pat. No. 7,602,947, the contents of which are incorporated herein by reference.

A camera is illustratively a digital camera wherein a visual image is captured on a charge coupled device (CCD) or a complementary metal oxide semiconductor (CMOS) image sensor. CCD or CMOS imaging detectors are known in the art. Either sensor is illustratively available from Dalsa Corp., Billerica, Mass. Closed Circuit Television cameras with either detector are available to those of skill in the art illustratively from Lot Oriel Corp., Palaiseau, France. A camera is optionally operated in single shot mode wherein a user is able to select and capture a single digital image, or in continuous mode.

A microprocessor is coupled with or present in a camera for initial processing of an image. One such processor is the MPC5121e 3D facial recognition processor available from Freescale Semiconductor Co., Austin, Tex., the S3C2410A processor, or other processors known in the art. An illustrative microprocessor system operable in the present invention is described by Sun, Y, et al., An Embedded System of Face Recognition Based on ARM and HMM, in Lecture Notes in Computer Science, Springer Berlin/Heidelberg, 2007, ISSN 0302-9743, the contents of which are incorporated herein by reference.

A microprocessor is optionally equipped with facial recognition and biometric data generating software. Illustrative software illustratively includes the Face Detection SDK produced by Ayonix Co., Ltd., Osaka City, Japan.

An inventive system optionally includes a lighting system at or near the location of the target to be imaged by the camera. A lighting system operable is illustratively described in U.S. Pat. Application No. 2009/0251560, the contents of which are incorporated herein by reference.

Various aspects of the present invention are illustrated by the following non-limiting examples. The examples are for illustrative purposes and are not a limitation on any practice of the present invention. It will be understood that variations and modifications can be made without departing from the spirit and scope of the invention.

### Example 1

Cognitive Engrams Extracted from Data of Existing Studies

Recent scientific publications utilizing fMRI to study neurocognitive functions which detail the specific coordinates of activation areas for visual objects were selected, and are described herein. Three-dimensional graphical surface, bubble point or other visual or mathematical representations of the activation maps are constructed by plotting the xyz coordinates. The xyz coordinates were supplied by all three sets of investigators using the normalized space of the Talairach and Tournoux brain atlas (1988), the contents of which are incorporated herein by reference.

All of the publications whose data is reprocessed presented test objects to test subjects while lying in an MRI scanner, using a flat panel display situated to allow visibility during scanning.

Bernard et al. (2004), incorporated herein by reference, studied the presentation and processing of visual imaging of famous and non-famous faces. The study used an event-related fMRI design, utilized a 4-T MRI system, with stimuli consisting of 80 black and white pictures of adult faces, half of famous and half of non-famous people. The xyz coordinates of the activation areas were supplied in Table 2 of Bernard et al. When the three-dimensional representation of Bernard et al. is prepared the following cognitive engrams are determined.

When Bernard 2004 data is processed to create three-dimensional activation maps two cognitive engrams are developed: one for famous and one for non-famous faces. It is apparent that by viewing these activation patterns that data specific to famous versus non-famous faces is obtained.

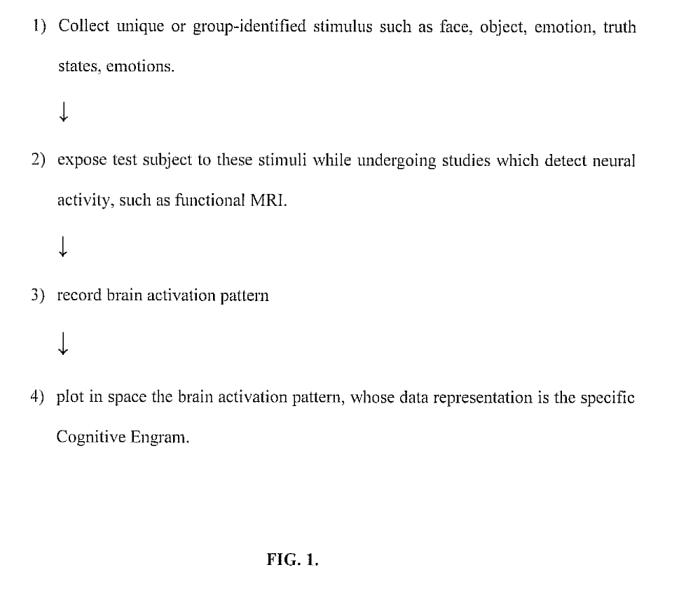

The Bernard et al. data is processed into cognitive engrams and demonstrate the potential to distinguish whether specific faces are recognizable, and whether they are famous or non-famous. Differences between three classes of objects (face, chair, house) and familiar and non-familiar words can also be distinguished by the pattern of the cognitive engrams generated. With the development of more detailed activation maps, from a larger number of individuals, with a more detailed graphical representation of concepts, a Rosetta Database forming a database of cognitive engrams for individual faces, objects, places, smells, emotions and concepts (hate, intent to deceive, pain, etc.) is developed. Such a database provides two-way interpretation of cognitive engrams allowing the identification of the presence of specific thoughts (persons, places, intents) in an individual's brain. A schematic diagram of an inventive process to determine a cognitive engram is provided in FIG. 1.

### Example 2

### Distinguishing Cognitive Engrams of Standard Faces

Healthy adult volunteers consent to an IRB-approved clinical study protocol generally as described by Marks D H, Adineh M, Gupta S: Determination of Truth from Deception Using Functional MRI and Cognitive Engrams. The Internet Journal of Radiology [peer-reviewed serial on the Internet]. 2006. Volume 5, Number 1; incorporated herein by reference. Continual neuroimaging (fMRI) was performed during viewing of the test stimuli to capture structural and functional data. The data was analyzed for the presence of neuroimaging activation shown to correspond to cognition and visual recognition.

Imaging data were collected in a 3-T General Electric Signa Excite seamier with whole head coil. Changes in the blood oxygen level dependent BOLD T2*-weighted MRI signal were measured using a gradient-echo echoplanar sequence. In each time series 18 (128.times.128) or 28 (64.times.64) contiguous axial slices were obtained. Three sets of scans are performed per experiment: localizer; MRI for functional data; and high resolution anatomic.

The subject lies in the MRI scanner, wearing a phased array MRI head coil as part of the regular head scan. Mounted on the head coil is a 45 degree mirror, allowing them to see down toward their feet and view the test images. Optionally, other methods of presentation of the stimulus can be used, such as video feed via fiber optic cable, sound, odors, tactile, etc. Displayed near the bottom of the subject's feet is a large photo (for photo presentation), projected image, or a white blank paperboard, which can have a large X marked across it (to center the observer's concentration), which is totally within the field of view of the test subject lying in the scanner. Other means of presentation of visual test images include a goggle image projection set, and use of an MRI-compatible video monitor.

The functional MRI scans last between 110 to 220 seconds total; after a 6 second lead-in time, the blank is displayed for 6 to 8 seconds, then the picture for 6 to 8 seconds, blank, picture, for a total of 5 to 10 repetitions. Other presentation sequences and study designs can be used.

In a separate scan, high-resolution full volume structural images are obtained for each subject, using fast SPGR imaging (146, 1.0-mm thick axial slices, no spaces, TR=8, TE=3.2, FOV=24 cm, 256.times.256 matrix). These T1-weighted images provide detailed anatomical information for registration and three-dimensional normalization to the Talairach and Tournoux atlas (1988), as described below.

There are approximately eight runs per person for these first few scans:
TABLE-US-00001 Run Process 1 Localizer (sets up scanner field, directs scanned areas into brain, avoids “ghost” images) 2 First photo 3 Second photo 4 Third photo (if used) 5 Detailed Anatomic run

### Data Analysis

Imaging data is analyzed in two ways: conventional and raw data. Conventional analysis refers to the generation of activation maps correlated with anatomical representative images. Modeling of raw imaging data involves the manipulation of the basic output from the scanner itself, using techniques such as neural networks. Voxels can be individually compared with the properties of intensity of activation and corrected spatial localization.

The following paragraphs explain an analysis procedure. The fMRI scan volumes are motion-corrected and spatially smoothed in-plane. MRI data files are analyzed using programs such as MedX to determine the location of activated voxels—defined as voxels (volumetric areas of the brain, within the imaging field) from a brain region that respond differentially to the test (visual) stimuli. Voxels are selected that show an overall increase in activity for meaningful stimuli, for example, a positive regression weight for the contrast between a test photo and control (blank page) stimuli. I used an uncorrected probability >0.05, meaning every voxel showing activation with the probability of more than 95% is selected in the analysis. Other probability levels can be used.

Activated voxels are then segregated into clusters according to the category of objects that evoke the maximal response. For conventional analyses, clusters of seven or more contiguous voxels are typically considered significant, but other types of data analysis can use more or less. In separate analyses, activated voxel data can also be parsed on the basis of degree of activation.

The anatomical locations of clusters of voxels showing significant differences between responses are determined by superimposing the statistical maps on coplanar high-resolution structural images. The partial volume structural images are registered with the full volume high-resolution images using programs such as Automated Image Registration (Woods, Mazziotta & Cherry, 1993, the contents of which are incorporated herein by reference).

The full volume high-resolution images are normalized to the Talairach and Tournoux atlas (1988) using programs like MedX. Both transformations (registration and normalization) are then applied to the statistical maps in order to obtain the Talairach coordinates of brain regions that respond maximally to the test stimuli to generate a cognitive engram.

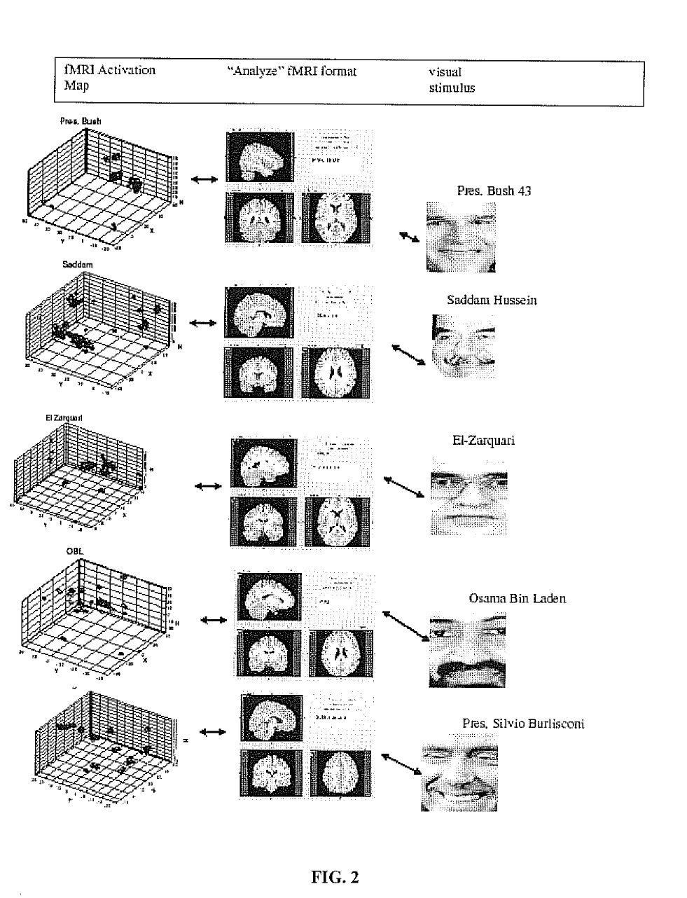

Functional MRI experiments are performed on normal subjects while they are looking at photos. As an example, the following activation map data points are created when viewing photos of President Bush, as detailed in Table 1. Correlation between fMRI activation noted in DICOM images (center) with the three-dimensional activation map—the Cognitive Engram, and the initiating brain stimulation (photo of President Bush) is shown in FIG. 2.

TABLE-US-00002 TABLE 1 Brain Activation Points for Photo of President Bush Location X Y Z Right Cerebrum, Limbic Lobe, Parahippocampal Gyrus, Gray 32 -20 -14 Matter, Hippocampus Left Cerebrum, Temporal Lobe, Inferior Temporal Gyrus, Gray -44 -16 -28 Matter, Brodmann area 20 Left Cerebrum, Temporal Lobe, Fusiform Gyrus, White Matter, * -42 -16 -26 Left Cerebrum, Temporal Lobe, Fusiform Gyms, White Matter, * -45 -16 -26 Left Cerebrum, Temporal Lobe, Inferior Temporal Gyrus, White -44 -18 -28 Matter, * Left Cerebrum, Temporal Lobe, Inferior Temporal Gyrus, White -44 -18 -30 Matter, * Left Cerebrum, Temporal Lobe, Inferior Temporal Gyrus, White -45 -18 -28 Matter, *

### Example 3

### Sample Interrogation Protocol

Background information is obtained on the subject's name, age, sex, SSN, birth city, medications, current and past illnesses, psychological history, criminal history, accusation, etc. Then a basic set of standard images is presented to the test subject while undergoing neuroimaging in order to obtain a background pattern of activation. Individual responses to questions are recorded via a number of techniques including verbal responses and pressing of buttons on handgrips. The test subject is presented with between ten to twenty test questions and asked to supply intentionally deceptive and intentionally truthful responses to each question.

The test subject is presented case-specific questions with accompanying images/displays of words and phrases concerning: Facts about their own personal life, name, sex, age, SSN, residence, criminal history; and Specific photos/questions/statements relating to the subject at issue, including potentially the specific crime, intention, conspiracy collaborators, locations, objects.

The raw imaging data (structure and function) is analyzed and three-dimensional activation and anatomical maps are created. The pattern of activation is correlated with specific responses to images and concepts, as part of a correlative dictionary (Rosetta Database) between three-dimensional fMRI activation maps and concepts/images presented.

The activation map is analyzed according to Example 2 to create the optimum correlation between activation areas and truthful/deceptive responses on control and question images/concepts. Using the internal controls on individual patients, and accumulated group controls, an interpretation on truthfulness is made for:

responses to individual questions; and

recognition of individual presented images and concepts.

### Example 4

Use of fMRI to Predict Neurologic Adverse Effects of Interferon Used to Treat Patients with Hepatitis C

Brain activation cognitive engrams of individuals who have been selected to receive interferon therapy are produced, before and after 4-6 weekly injections of interferon. Standard medical questioning and written evaluation tools of depression such as Beck Depression Scale are employed at baseline and weekly to detect the development of INF-associated depression. Individuals who require treatment of interferon-induced depression (receive an antidepressant such as SSRI medication) undergo repeat fMRI after an appropriate therapeutic interval in order to evaluate changes in activation map which can correlate with a therapeutic antidepressive response.

Immediate follow-up studies include dose-response of interferon to fMRI indices of clinical depression and depressive response as a side effect of antidepressants as shown by fMRI indices of depression. The immediate benefit of this is to develop an objective method of predicting a depressive response as a side effect of essential medications. The response predictive protocol is provided in Table 2.

TABLE-US-00003 TABLE 2 Interferon Study Design 1 Pre-Protocol Eligibility Screening 2 Eligible patient MRI and fMRI Beck Depression Scale 3 Week 1 Begin hepatitis C treatment, including interferon 4 Week 4-6, or sooner if significant MRI and fMRI Beck adverse CNS symptoms develop Depression Scale 5 If CNS symptoms, but are not severe enough to discontinue interferon, begin an antidepressant 6 2-6 weeks antidepressant MRI and fMRI Beck Depression Scale

The study subjects undergo continual MRI neuroimaging while viewing test stimuli in order to capture structural and functional data as detailed in Example 2. These data are analyzed for the presence of neuroimaging activation previously shown to correspond to cognition and depression.

Test visual stimuli are chosen to induce a strong depressive response in the viewer.

A normal test subject is shown photos of President Bush, Saddam Hussein, and of a grieving woman kneeling at a grave as detailed in Example 2. The photo of the grieving woman is used to generate a feeling of sadness. This subject shows activation in the right inferior and middle temporal gyrus and in the fusiform gyrus areas known to correlate with the provocation of sadness in normal individuals. This activation is only noted when viewing the grieving woman—not for the test photos of President Bush and Saddam Hussein. Individuals scoring as depressed on the Beck Depression Scale show varying degrees of activation in these cerebral regions for all three images.

### Example 5

Associating fMRI with Biometric Data Obtained from Facial Recognition Software

Data is coordinated between brain activation obtained from fMRI while a subject is viewing specific visual stimuli as detailed in Example 2 and biometric data obtained using biometric facial recognition software (Ayonix Co., Ltd., Osaka City, Japan).

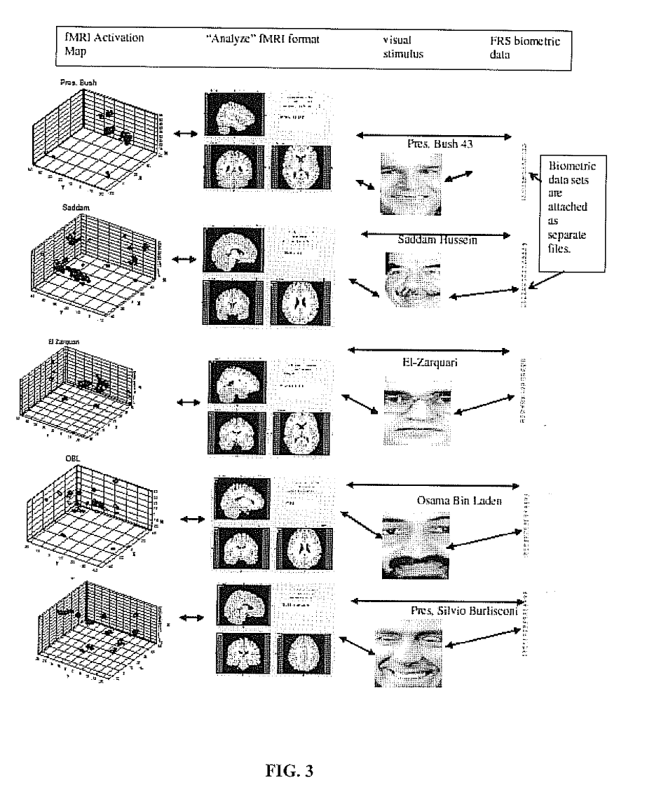

The following sequences are used. Continuous fMRI scans are taken for 110 seconds each. EPI parameters are: TE 35, TR 2000, multiphase screen, 55 phases per location, interleaved, flip angle 90, delay after acquisition-minimum. Using a visual stimulus package color photographs are presented in a mini-block design while neuroimaging is performed. In a typical session, after a 4 second lead-in time, a blank screen is displayed for 4 seconds, then the picture of interest for 4 seconds, and this is repeated for the scan time. Illustrated is the central Rosetta Database, which stores a library of brain stimuli such as specific pictures, videos, sounds, concepts, emotions, etc. Associated with each specific stimulus is associated either the fMRI activation map—the cognitive engram, the associated biometric data set, or both data sets. This allows a lookup function, whereby knowing the fMRI pattern, or the biometric dataset, allows the determination of the specific stimulus, such as a specific face, as shown in FIG. 3.

The fMRI scan volumes are motion-corrected and spatially smoothed in-plane. MRI data files are normalized and analyzed using MedX (version 3.4.3, Sensor Systems, Sterling, Va.) to compute statistical contrasts and create a map representing significantly activated areas of the brain that respond differentially to a visual test stimuli.

For the voxels that show an overall increase in activity for meaningful stimuli, a positive regression analysis for the contrast between a test photo and control (blank page) stimuli is performed creating an activation map containing specific voxels with an uncorrected probability, P.ltoreq.0.05; meaning every voxel showing activation with the probability greater than 95%. Only those voxels with 95% or greater probability are selected for further analyses. The statistical map is then superimposed on coplanar high-resolution structural images. The partial volume structural images are registered with the full volume high-resolution images using Automated Image Registration (Woods 1993).

The full volume high-resolution images are then transformed (registered and normalized) to the Talairach and Tournoux atlas (1988) using MedX tools. Each activated voxel on these images is selected to obtain Talairach coordinates of brain regions that respond maximally to the test stimuli and to further generate a Cognitive Engram. Observed patterns of activation are associated with the nature of the response: truthfulness or deception.

Three-dimensional graphical representations of the identified activation maps are constructed by plotting the xyz coordinates, using the program DPlot (HydeSoft Computing, Vicksburg, Miss.).

The same photographs of faces used to prepare fMRI data are introduced to the Ayonix biometric system by scanning the photograph or directly inputting the digital photograph into the software and biometric face data sets are generated. Methods of converting images into biometric information are illustratively described in JP2009237669, the contents of which are incorporated herein by reference. Generally, the biometric face recognition system includes a registered person face image data storage device for storing a plurality of pieces of facial feature information data for each of a plurality of oblique direction angles for each of facial image data of at least two registrants, A standard human face image data storage device for storing information on an output threshold value for each light and dark rectangular filter for each of a plurality of predetermined inclination angles in a bright/dark rectangular filter having a predetermined characteristic, Face detection means for extracting human face area image data from the image data and a plurality of characteristic information on the face of the extracted human face area image data and sets them as a plurality of face characteristic information data Facial feature extraction means for extracting a plurality of facial feature information data from facial feature image data of each registered person stored in registered facial image data storage means And a specifying unit for comparing the number of facial feature information data with the number of facial feature information data to specify a corresponding registrant, and the face detecting unit is previously determined for the detection target area image data predetermined in the target image data Means for sequentially applying a bright/dark rectangular filter having a characteristic to the reference axis direction of the target image data at a plurality of predetermined inclination angles to acquire a bright/dark rectangular filter output value for each inclination angle; Means for comparing the brightness/dark rectangular filter output value for each inclination angle with the bright/dark rectangular filter output threshold for each inclination angle stored in the standard person's face image data storage device, and means for comparing the bright/dark rectangular filter output threshold Means for extracting the detection target region image data as candidate face region image data when there is the inclination angle of the light and dark rectangular filter which is the brightness/dark rectangle filter output value, Regarding the candidate face area image data that is output, it is judged whether or not the skin color of the person is detected, and when the flesh color of the person is detected, the candidate face area image data is extracted as human face area image data Means for generating a signal. The face recognition software is capable of reducing the false recognition rate of face recognition.

Furthermore, the biometric face recognition system stores, for each of a plurality of predetermined rectangular filtering areas, image data of a face of a person as a standard, regarding an output threshold value for a plurality of light and dark rectangular filters Store information. Then, with respect to the candidate face region image data, a bright/dark rectangle filter is further sequentially applied with a plurality of rectangular filtering areas to acquire the bright/dark rectangular filter output for each rectangular filtering area, and this is stored in the standard person face image data storage device When compared with the stored brightness/dark rectangular filter output threshold value for each rectangular filtering area and as a result of the comparison, if there is a filtering area portion of the light/dark rectangle filter which is the light/dark rectangle filter output value less than the light/dark rectangular filter output threshold value, the filtering The area part is deleted from the candidate face region image data to narrow down the candidate face region image data. As a result, the size of the region of the candidate face region image data can be minimized.

The biometric face recognition process using the Ayonix software is done in a frequency domain with exponential functions, the data sets can vary (0.1%-5% changes are observed) somewhat for the same pictures. The Ayonix software does not use Local Feature analysis, so there is no direct representation in the number set of how the eyes, nose, ears and mouth are arranged spatially. The Gabor algorithm extracts the overall facial features in specific points, then the Ayonix software checks the high frequency on the contour points. According to that information, the distance between eyes, nose, mouth is calculated by using triangular relation. Then, the grayscale level is checked. For example, eyes of Japanese people give lower values than for European people since their eye bones are less prominent in appearance. The size and shape of the nose bridge and its ratio with mouth are measured. The data is all done in frequency domain and represented as a result of FFT and Wavelet. Skin color is not taken into account with the Ayonix biometric software, as the image is transferred to grayscale before processing.

FIG. 3 illustrates brain activation data obtained from fMRI while viewing specific visual stimuli associated with the biometric data obtained using facial recognition software obtained from a security camera. In this way, fMRI from an individual is used to construct an image of the person they saw without resort to inaccuracies created through the use of an artist working under the direction of the individual who saw the person. Correlation between fMRI activation noted in DICOM images (center) with the three-dimensional activation map (left)—the Cognitive Engram, the initiating brain stimulation (photo of President Bush), and the biometric analysis of the photo (far right).

### Example 6

### Camera Processed Facial Recognition

Security monitoring cameras commonly observe persons whose identity is requested. The video or photograph signal is transferred from the camera via cable or signal to a computer for subsequent processing. At the remote computer a number of steps are taken to convert the camera's electronic image to a biometric template which is viewed and compared to a library of other faces for further identification.

The invention described herein, in contrast, provides a camera with built-in microprocessing and transmitting capabilities that allow the image to be initially processed within the camera unit and those partially processed biometric facial data are then transmitted to a remote site where the imaging data is further processed. This dramatically reduces the amount of data transmitted and bandwidth required for surveillance or other uses. An example of such a system is schematized below.

### Hardware

I. CMOS CCT high resolution cameras are provided with the following integral equipment: A. Microprocessor, with capability to: a. localize faces, b. localize eyes within face, c. password encoding and compression of data. d. additional operations performed by the microprocessor integrated into or attached to the camera: i. quality control of image analysis, normalization of face, preprocessing of data, identification of essential facial features, rotation and extraction, preparation of biometric template. B. Transmission unit to allow transfer of data via internet, wifi, Bluetooth, direct cable, radio waves, and other communication methods.

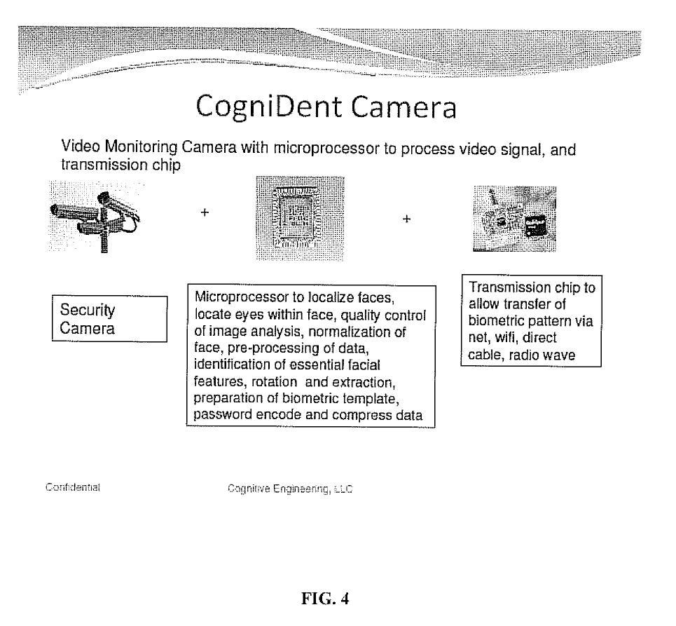

FIG. 4 illustrates data transmitted from a single camera to a remote or local computer for subsequent comparison and identification. Photographs of George Bush, Saddam Hussein, and other individuals of relative levels of popular recognition are entered into the camera wherein facial recognition and biometric data are generated for each face.

Data is processed directly within the monitoring camera unit followed by transmission of biometric data to a central site for comparison to a database and optional identification against a database stored data set. This system frees individual monitoring sites of the requirement for having a computer, database, or analytic software immediately near each monitoring camera and dramatically reduces the amount of data transmitted improving existing bandwidth usage and allowing more monitoring stations to use the same bandwidth.

The following references and those recited in the specification are incorporated herein by reference for the entirety of their teaching.

### REFERENCES

Beauchamp M H et al. Dynamic functional changes associated with cognitive skill learning of an adapted version of the Tower of London task. Neuroimage. 2003 November; 20(3): 1649-60.
Bernard F A et al. The hippocampal region is involved in successful recognition of both remote and recent famous faces. NeuroImage 22 (2004) 1704-1714.
Binder J R et al. Conceptual Processing During the Conscious Resting State: A functional MRI Study. J Cog Neuroscience 11(1):80-93, 1999.
Binder J R et al. Neural Correlates of Lexical Access during Visual Word Recognition. Journal of Cognitive Neuroscience 15:3, pp. 372-393, 2003.
Blair. R J R et al. Dissociable neural responses to facial expressions of sadness and anger. Brain (1999), 122, 883-893.
Blaizot X. et al. Mapping the visual recognition memory network with PET in the behaving baboon. J Cereb Blood Flow Metab. 2000 February; 20(2):213-9.
Bloom A S et al. Determination of Drug-Induced Changes in Functional MRI Signal Using a Pharmacokinetic Model. Hum Brain Mapping 8:235-244, 1999.
Buhmann C et al. Pharmacologically modulated fMRI-cortical responsiveness to levodopa in drug-naive hemiparkinsonian patients. Brain 126, 451-461, 2003.
Canli T et al. Brain activation to emotional words in depressed vs healthy subjects. Brainimaging Neuroreport. Vol 15, No 17, 3 Dec. 2004 2585.
Chao L L, Haxby J V, Martin A. Attribute-based neural substrates in temporal cortex for perceiving and knowing about objects. Nat Neurosci. 1999 October; 2(10):913-9.
Chua P et al. A functional anatomy of anticipatory anxiety. NeuroImage 9: 563-571, 1999.
Courthey S M, Petit L. Haxby J V, et al. The role of prefrontal cortex in working memory: examining the contents of consciousness. Philos Trans R Soc Lond B Biol Sci. 1998 Nov. 29; 353(1377):1819-28.
Critchley H D, Elliott R, Mathias C J, Dolan R J. Neural activity relating to generation and representation of galvanic skin conductance responses: A functional magnetic resonance imaging study. J. Neurosci. 20: 3033-3040, 2000.
Dougherty D D, Rauch S L, Jenike M A. (2004). Pharmacotherapy for Obsessive-Compulsive Disorder. JCLP/In Session 60, 1195-1202.
Dodson C S, Johnson M K. Rate of false source attributions depends on how questions are asked. Am J. Psychol. 1993 Winter; 106(4):541-57.
Fossati P, Radtchenko A, Boyer P. (2004). Neuroplasticity: from MRI to depressive symptoms. European Neuropsychopharmacology 14, S503-S510.
Fan J et al. Functional magnetic resonance imaging of source versus item memory. Neuroreport. 2003 Dec. 2; 14(17):2275-81.
Fiebach C J, Friederici A D. Processing concrete words: fMRI evidence against a specific right-hemisphere involvement. Neuropsychologia. 2004; 42(1):62-70.
Fletcher P C, Henson R N. Frontal lobes and human memory: insights from functional neuroimaging. Brain (2001 May) 124(Pt 5):849-81.
Fossati P et al. Neuroplasticity: from MRI to depressive symptoms. European Neuropsychopharmacology 14 (2004) S503-S510.
Fossati P et al. In search of the emotional self: an FMRI study using positive and negative emotional words. Am J. Psychiatry. 2003 November; 160(11):1938-45.
Fu C H et al. Attenuation of the neural response to sad faces in major depression by antidepressant treatment: a prospective, event-related functional magnetic resonance imaging study. Arch Gen Psychiatry. 2004 September; 61(9):877-89.
Fujii T et al. Encoding-related brain activity during deep processing of verbal materials: a PET study. Neurosci Res Vol. 44 2002 December pp. 429-38.
Gaillard W D, Hertz-Pannier L, Mott S H, Barnett A S, LeBihan D, Theodore W H. Functional anatomy of cognitive development: fMRI of verbal fluency in children and adults. Neurology (2000 Jan. 11) 54(1):180-5.
Gerlach C et al. Perceptual differentiation and category effects in normal object recognition: a PET study. Brain (1999 November) 122 (Pt 11):2159-70.
Gevins A et al. Electroencephalographic imaging of higher brain function. Philos Trans R Sac Lond B Biol Sci. 1999 Jul. 29; 354(1387):1125-33.
Gonsalves B et al. Neural evidence that vivid imagining can lead to false remembering. Psychological Science 15(10): 655-660, 2004.
Gross J et al. Properties of MEG tomographic maps obtained with spatial filtering. Neuroimage. 2003 August; 19(4):1329-36.
Gur R C, Gur R E, Resnick S M, Skolnick B E, Alavi A, Reivich M. The effect of anxiety on cortical cerebral blood flow and metabolism. J. Cereb. Blood Flow Metab. 7: 173-177, 1987.
Hagoort P. How the brain solves the binding problem for language: a neurocomputational model of syntactic processing. Neuroimage. 2003 November; 20 Suppl 1:S18-29.
Hammers A et al. Three-dimensional maximum probability atlas of the human brain, with particular reference to the temporal lobe. Hum Brain Mapp. 2003 August; 19(4):224-47.
Hartley A A, Speer N K. Locating and fractionating working memory using functional neuroimaging: storage, maintenance, and executive functions. Microsc Res Tech. 2000 Oct. 1; 51(1):45-53. [0154] Haxby J V, Gobbini M I, Furey M L et al. Distributed and Overlapping Representations of Faces and Objects in Ventral Temporal Cortex. Science Vol 293 28 Sep. 2001 2425.
Haxby J V et al. Object-form topology in the ventral temporal lobe. Response to I. Gauthier (2000). Trends Cogn Sci. 2000 January; 4(1):3-4.
Henson R N. Neuroimaging studies of priming. Prog Neurobiol. 2003 May; 70(1):53-81.
Ishai A et al. Distributed representation of objects in the human ventral visual pathway. 1999 PNAS. 96:9379-9384.
Ishai A et al. The representation of objects in the human occipital and temporal cortex. (2000) Journal of Cognitive Neuroscience, 12 Supplement 2, pp. 35-51.
Jernigan T L et al. More “mapping” in brain mapping: statistical comparison of effects. Hum Brain Mapp. 2003 June; 19(2):90-5.
Johnson M K, Hashtroudi S, Lindsay D S. Source monitoring. Psychological Bulletin 114:3-28, 1993.
Juengling, F D et al. Prefrontal cortical hypometabolism during low-dose interferon alpha treatment. Psychopharmacology 152, 383-389, 2000.
Kim D S, Kim M, Ronen I et al. In vivo mapping of functional domains and axonal connectivity in cat visual cortex using magnetic resonance imaging. Magn Reson Imaging. 2003 December; 21(10):1131-40.
Knight D C et al. Neural substrates mediating human delay and trace fear conditioning. J Neurosci. 2004 Jan. 7; 24(1):218-28.
Koechlin E, Ody C, Kouneiher F. The architecture of cognitive control in the human prefrontal cortex. Science. 2003 Nov. 14; 302(5648):1181-5. Comment in: Science. 2003 Nov. 14; 302(5648):1133 PMID: 14615503.
Koechlin E et al. The architecture of cognitive control in the human prefrontal cortex. Science. 2003 Nov. 14; 302(5648):1181-5.
Kozel A, Padgett T M, George M S. A Replication Study of the Neural Correlates of Deception. Behavioral Neuroscience, Vol. 118, No. 4, 2004.
Lange N. What can modern statistics offer imaging neuroscience? Stat Methods Med Res. 2003 October; 12(5):447-69.
Langleben D D et al. Brain Activity during Simulated Deception: An Event-Related Functional Magnetic Resonance Study. NeuroImage 15, 727-732 (2002).
Lee T M et al. Lie Detection by Functional Magnetic Resonance Imaging. Hum. Brain Mapping 15:157-164, 2002.
Lykken, D T. 1991. Why (some) Americans believe in the lie detector while others believe in the guilty knowledge test. Integr. Physiol. Behay. Sci. 26: 214-222.
Matthews, S C, Paulus, M P, Dimsdale, J E. (2004). Contribution of functional neuroimaging to understanding neuropsychiatric side effects of interferon in Hepatitis C. Psychosomatics 45, 281-286.
McGonigle D J et al. Variability in fMRI: An examination of intersession differences. NeuroImage, 11, 708-734, 2000.
Lanius R A et al. The nature of traumatic memories: a 4-T FMRI functional connectivity analysis. Am J Psychiatry. 2004 January; 161(1):36-44.
Lanius R A, Williamson P C, Densmore M et al. The nature of traumatic memories: a 4-T FMRI functional connectivity analysis. Am J Psychiatry. 2004 January; 161(0:36-44.
Marshall J C, Fink G R. Cerebral localization, then and now. Neuroimage. 2003 November; 20 Suppl 1:S2-7.
Maldjian J A et al. An automated method for neuroanatomic and cytoarchitectonic atlas-based interrogation of fMRI data sets. Neuroimage. 2003 July; 19(3):1233-9.
McGonigle D J et al. Variability in fMRI: An examination of intersession differences. NeuroImage, 11, 708-734, 2000.
Michael D et al. The neural basis of episodic memory: evidence from functional neuroimaging. Phil. Trans. R. Soc. Lond. B (2002) 357, 1097-1110 1097.
Mungas D et al. MRI predictors of cognition in subcortical ischemic vascular disease and Alzheimer's disease. Neurology (2001 Dec. 26) 57(12):2229-35.
Na, D G et al. Functional MR imaging of working memory in the human brain. Korean J Radiol 2000 January-March pp. 19-24.
Nielsen F A et al. Mining for associations between text and brain activation in a functional neuroimaging database. Neuroinformatics. 2004; 2(4):369-80.
Nyberg L et al. Brain imaging of human memory systems: between-systems similarities and within-system differences. Brain Res Cogn Brain Res, Vol. 13, 2002 April, pp. 281-92.
National Research Council. (2003). The polygraph and lie detection. Retrieved December, 2004, from the National Academies.
Ogawa S, Lee T M, Kay A R, Tank D W. Brain magnetic resonance imaging with contrast dependent on blood oxygenation. Proc Natl Acad Sci USA. 1990 December; 87(24):9868-72.
Okado Y and Stark C, JHU. Neural processing associated with true and false memory retrieval. Cognitive, Affective, & Behavioral Neuroscience. 2003, 3 (4), 323-334.
Olesen P J et al. Increased prefrontal and parietal activity after training of working memory. Nat Neurosci. 2004 January; 7(1):75-9.
Papadakis N G et al. A measure of curve fitting error for noise filtering diffusion tensor MRI data. J Magn Reson. 2003 September; 164(1):1-9.
Pietrini P, Furey M L, Ricciardi E, et al. Beyond sensory images: Object-based representation in the human ventral pathway. Proc Natl Acad Sci USA. 2004 Apr. 13; 101(15):5658-63. Epub 2004 Apr. 2.
Prabhakaran V, Narayanan K, Zhao Z et al. Integration of diverse information in working memory within the frontal lobe. Nat Neurosci. 2000 January; 3(1):85-90.
Reber P J et al. Neural correlates of successful encoding identified using functional magnetic resonance imaging. J. Neuroscience. 22:9541-9548, 2002.
Robertson L T. Memory and the brain. J Dent Educ. 2002 January: 66(1):30-42.
Rugg M D, Often L J, Henson R N. The neural basis of episodic memory: evidence from functional neuroimaging. Phil. Trans. R. Soc. Lond. B (2002) 357, 1097-1110.
Rueckert D, Frangi A F, Schnabel J A. Automatic construction of 3-D statistical deformation models of the brain using nonrigid registration. IEEE Trans Med Imaging. 2003 August; 22(8):1014-25.
Salmeron B J, Stein E A. Pharmacological applications of magnetic resonance imaging. Psychopharmacol Bull (2002 Winter) 36(1):102-29.
Schmithorst V J, Holland S K. Comparison of three methods for generating group statistical inferences from independent component analysis of functional magnetic resonance imaging data. J Magn Reson Imaging. 2004 March; 19(3):365-8.
Szaflarski J P, Holland S K et al. High-resolution functional MRI at 3T in healthy and epilepsy subjects: hippocampal activation with picture encoding task. Epilepsy Behay. 2004 April; 5(2):244-52.
Schmithorst V J et al. Cognitive functions correlate with white matter architecture in a normal pediatric population: A diffusion tensor MRI study. Hum Brain Mapp. 2005 Apr. 27.
Slotnick S D and Schacter D L. A sensory signal that distinguishes true from false memories. Nature Neuroscience 7(6):664-669, 2004.
Shallice T. Functional imaging and neuropsychology findings: how can they be linked? Neuroimage. 2003 November; 20 Suppl 1:5146-54.
Smejkal V, Druga R, Tintera J. Olfactory activity in the human brain identified by fMRI. Bratisl Lek Listy. 2003; 104(6):184-8.
Smith C D, Andersen A H, Kryscio R J et al. Altered brain activation in cognitively intact individuals at high risk for Alzheimer's disease. Neurology (1999 Oct. 22) 53(7):1391-6.
Sobottka S B et al. Comparison of functional brain PET images and intraoperative brain-mapping data using image-guided surgery. Comput Aided Surg. 2002; 7(6):317-25.
Stuss D T, Gallup G G, Alexander M P. The frontal lobes are necessary for “theory of mind”. Brain (2001 February) 124(Pt 2):279-86.
Suppes P and Han B. Brain-wave representation of words by superposition of a few sine waves. Proc Natl Acad Sci USA. 2000 Jul. 18; 97(15): 8738-8743.
Talairach J and Tournoux P. (1988). Co-planar stereotaxis atlas of the human brain (M. Rayport, Trans.). New York: Thieme Medical.
Thomas K M. Assessing brain development using neurophysiologic and behavioral measures. J Pediatr. 2003 October; 143(4 Suppl):546-53.
Vythilingam M et al. Hippocampal Volume. Memory, and Cortisol Status in Major Depressive Disorder: Effects of Treatment. Biol. Psychiatry 56, 101-112, 2004.
Wood E R, Dudchenko P A, Robitsek R J, Eichenbaum H. Hippocampal neurons encode information about different types of memory episodes occurring in the same location. Neuron (2000 September) 27(3):623-33.
Wagner A D, Koutstaal W, Schacter D L. When encoding yields remembering: Insights from event-related neuroimaging. Philas. Transact. Royal Soc London. B. Biological Sciences. 354:1307-1324, 1999.
Woods R P, Mazziotta J C, Cherry S R. MRI-PET registration with automated algorithm. J Comput Assist Tomogr. 1993 July-August; 17(4):536-46.
Wyland C L, Kelley W M, Macrae C N, Gordon H L, Heatherton T F. Neural correlates of thought suppression. Neuropsychologia. 2003; 41(14):1863-7.
Yin J C. Location, location, location: The many addresses of memory formation. Proc. Natl. Acad. Sci. USA Vol. 96, pp. 9985-9986, August 1999.
Yang Y. Gu H, Stein E A (2004). Simultaneous MRI Acquisition of Blood Volume, Blood Flow, and Blood Oxygenation Information during Brain Activation. Magnetic Res. Medicine 52, 1407-1417.
Zhang J X, Leung H C, Johnson M K. Frontal activations associated with accessing and evaluating information in working memory: an fMRI study. Neuroimage. 2003 November; 20(3):1531-9.
Zeineh M M, Engel S A, Thompson P M, et al. Dynamics of the hippocampus during encoding and retrieval of face-name pairs. Science 299, 24 Jan. 2003, pp 577-580.
Marks D H, Adineh M, Gupta S: Determination of Truth from Deception Using Functional MRI and Cognitive Engrams. The Internet Journal of Radiology [peer-reviewed serial on the Internet]. 2006. Volume 5, Number 1.
Marks D H, Adineh M, Wang B, Gupta S, Udupa J K. Multidimensional Representation of Concepts as Cognitive Engrams in the Human Brain. The Internet Journal of Neurology [peer reviewed serial on the Internet]. 2007. Volume 6, Number 1.
Marks D H, Adineh M. Wang B, Gupta S. Use of fMRI to Predict Psychiatric Adverse Effects of Interferon Treatment for Hepatitis C. Neuropsychiatric Disease and Treatment. 2007:3(5) 655-667.
Marks D H, Adineh M, Gupta S. MR Imaging of Drug-Induced Suicidal Ideation, Internet J Radiology [peer-reviewed serial on the Internet], 9(1). 2008.
Marks D H. Evaluation of Cognitive Impairment. Internet J Health. [peer-reviewed serial on the Internet]. 8(1), 2008.
Marks D H, Valsasina P, Rocca M and Filippi M. Case Report: Documentation of Acute Neck Pain in a Patient Using Functional MR Imaging. Internet Journal of Pain, Symptom Control and Palliative Care [peer-reviewed serial on the Internet]. Accepted, 2009.
Cox D D, Savoy R L. Functional magnetic resonance imaging (fMRI) “brain reading”: detecting and classifying distributed patterns of fMRI activity in human visual cortex. Neuroimage. 2003 June; 19(2 Pt 1):261-70.
Hammoud R I, Abidi B R, Abidi M A. Face Biometrics For Personal Identification: Multi-Sensory Multi-Modal Systems. Springer, 2007.

Articles on Face Biometrics
Haxby J V, Ishai I I, Chao L L, Ungerleider L G, Martin I I. Object-form topology in the ventral temporal lobe. Response to I. Gauthier (2000). Trends Cogn Sci. 2000 January; 4(1):3-4.
Kamitani Y, Tong F. Decoding the visual and subjective contents of the human brain. Nat Neurosci. 2005 May; 8(5):679-85.
Kay K N, Naselaris T, Prenger R J, Gallant J L Identifying natural images from human brain activity. Nature. 2008 Mar. 20; 452(7185):352-5.
Kriegeskorte N, Formisano E, Sorger B, Goebel R. Individual faces elicit distinct response patterns in human anterior temporal cortex. Proc Natl Acad Sci USA. 2007 Dec. 18; 104(50:20600-5.
Lee J H, Marzelli M, Jolesz F A, Yoo S S. Automated classification of fMRI data employing trial-based imagery tasks. Medical Image Analysis 13 (2009) 392-404.
Loffler G, Yourganov G, Wilkinson F, Wilson H R. fMRI evidence for the neural representation of faces. Nat Neurosci. 2005 October; 8(10):1386-90.
Maurer D, O'Craven K M, Le Grand R, Mondloch C J et al. Neural correlates of processing facial identity based on features versus their spacing. Neuropsychologia 45 (2007) 1438-1451.
Marks D H, Adineh M, Wang B, Gupta S, Udupa J K. Multidimensional Representation of Concepts as Cognitive Engrams in the Human Brain. The Internet Journal of Neurology [peer-reviewed serial on the Internet]. 2007. Volume 6, Number 1.
Marks, D H. Brain Function Decoding Process and System. U.S. Pat. No. 7,627,370.
Mur M, Bandettini P A, Kriegeskorte N. Revealing representational content with pattern-information fMRI—an introductory guide. Sac Cogn Affect Neurosci. 2009 Jan. 17.
Rotshtein P, Henson R N, Treves A, Driver J, Dolan R J. Morphing Marilyn into Maggie dissociates physical and identity face representations in the brain. Nat Neurosci. 2005 January; 8(1):107-13.
Rotshtein P, Geng J J, Driver J, Dolan R J. Role of Features and Second-order Spatial Relations in Face Discrimination, Face Recognition, and Individual Face Skills: Behavioral and Functional Magnetic Resonance Imaging Data. Cogn Neurosci. 2007a September; 19(9):1435-52.
Rotshtein P, Vuilleumier P, Winston J, Driver J, Dolan R. Distinct and Convergent Visual Processing of High and Low Spatial Frequency Information in Faces. Cereb Cortex, 2007b; 17(11):2713-24.
Thirion, B. et al. Inverse retinotopy: inferring the visual content of images from brain activation patterns. Neuroimage 33, 1104-1116 (2006).
Yamashita O, Sato M A, Yoshioka T, Tong F, Kamitani Y. Sparse estimation automatically selects voxels relevant for the decoding of fMRI activity patterns. Neuroimage. 2008 Oct. 1; 42(4):1414-29. Epub 2008 Jun. 6.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A computer-implemented process of correlating cognitive response comprising:
collecting remote biometric data with a digital security camera having a microprocessor coupled to said camera for modifying an image from said camera by:
localizing a face in the image,
localizing eyes within the face, and
compressing the remote biometric data before transmission;
collecting stimulated neural activity data from one or more subjects while said subject is exposed to a stimulus and mapping a neural activation in response to said stimulus onto neuronal structure of a subject brain;
constructing a unique three-dimensional cognitive engram being a multidimensional representation of neuroimaging data of neurophysiological changes that are responsible for storage of a specific memory element or concept in the subject brain representative of the stimulated neural activity data caused by said stimulus from said one or more subjects to a level of voxels;
compiling said cognitive engram in a database containing a plurality of engrams, each of said plurality of engrams associated with a different stimulus for interpreting cognitive response; and
correlating said unique three-dimensional cognitive engram stored in said database with the biometric data representative of said stimulus.

2. The process of claim 1 wherein collecting remote biometric data further comprises any of correcting face illumination, measuring face image quality, removing the localized face from the image, removing face image color information, arranging the localized facial features into a matrix array, and removing noise from the image.

3. The process of claim 1 wherein said subject is a human.

4. The process of claim 1 wherein said neural activity data are collected by magnetic resonance imaging, functional magnetic resonance imaging, magnetic resonance angiography, magnetic resonance spectroscopy, computerized tomography, positron emission tomography (SPECT), electroencephalogram, magnetic encephalogram, other forms of functional neuroimaging, or combinations thereof.

5. The process of claim 1 wherein said stimulated neural activity data are collected as a plurality of tomographic scans compiled as two-dimensional slices.

6. The process of claim 1 further comprising performing image noise removal from said stimulated neural activity data prior to constructing said cognitive engram.

7. The process of claim 1 further comprising performing an operation on said stimulated neural activity data prior to constructing said cognitive engram, said operation selected from the group consisting of: correcting for subject movement artifact, reprocessing sequential images, blood oxygen level dependency consideration and statistical deformation modeling.

8. The process of claim 1 further comprising harvesting subject movement artifact overlap data for said stimulated neural activity data prior to constructing said cognitive engram.

9. The process of claim 1 wherein said stimulated neural activity data are derived from functional magnetic resonance imaging.

10. The process of claim 8 wherein said functional magnetic resonance imaging is correlated with data obtained from positron emission tomography, computerized tomography, magnetic resonance angiography, magnetic resonance spectroscopy or combinations thereof.

11. The process of claim 1 wherein said stimulated neural activity data are derived from arterial spin labeling and dynamic susceptibility weighted contrast enhanced magnetic resonance imaging.

12. The process of claim 1 wherein said stimulated neural activity data are derived from magnetization transfer and diffusion-tensor magnetic resonance imaging with whole brain tissue histogram analysis.

13. A system for capturing biometric data comprising:
an electronic camera to capture an image or at least a portion of the face of a person;
a microprocessor coupled to said camera, said microprocessor processing said image to generate biometric data representative of said face.

14. The system of claim 13 wherein generating said biometric data comprises any of localizing a face in the image, localizing eyes within the face, correcting face illumination, measuring face image quality, removing the localized face from the image, removing face image color information, arranging the localized facial features into a matrix array, and removing noise from the image.

15. The system of claim 13 further comprising a database for storing said biometric data.

16. The system of claim 15 further comprising a computer in electronic communication with said database, said computer configured to compare said biometric data in said database to biometric data from said camera.

17. The system of claim 15 wherein said database includes a cognitive engram associated with said biometric data.

18. The process of claim 1 further comprising said microprocessor modifying the image by performing quality control of image analysis.

19. The process of claim 1 further comprising said microprocessor modifying the image by rotation and extraction of the face from the image.

20. The process of claim 1 further comprising said microprocessor modifying the image by preparation of biometric template.
